(19) United States
(12) Patent Application Publication
(10) Pub. No.: US 2010/0239528 A1
(43) Pub. Date: Sep. 23, 2010

(54) Treating or Ameliorating a Neuroinflammatory or Demyelinating Disorder with Prolactin and Interferon-Beta

(75) Inventors: Samuel Weiss, Calgary (CA); Wee Yong, Calgary (CA)

Correspondence Address: FISH & RICHARDSON P.C., P.O BOX 1022, Minneapolis, MN, 55440-1022, US

(73) Assignee: STEM CELL THERAPEUTICS CORP., Calgary, AB (CA)

(21) Appl. No.: 12/669,893

(22) PCT Filed: Jul. 18, 2008
(86) PCT No.: PCT/CA08/01322

§ 371 Date: Jun. 3, 2010

Related U.S. Application Data
(60) Provisional application No. 60/950,948, filed on Jul. 20, 2007.
(60) Provisional application No. 61/012,259, filed on Dec. 7, 2007.

(51) Int. Cl. A61K 38/22; A61K 38/21; A61P 29/00; A61P 25/00; A61P 37/02

(52) U.S. Cl. 424/85.6; 424/85.4

(57) ABSTRACT

Methods, compositions and kits for treating or ameliorating a disorder associated with neuroinflammation or de-myelination in a mammal are disclosed. Said methods, compositions and kits comprise prolactin, a prolactin-inducing agent, or a variant, analog or functional fragment of prolactin in combination with interferon-β.

### CROSS REFERENCE TO RELATED APPLICATIONS

This application claims the benefit of priority to U.S. Provisional Application Nos. 60/950,948, filed on Jul. 20, 2007, and 61/012,259, filed on Dec. 7, 2007, which are incorporated by reference herein in their entireties.

### BACKGROUND

Disorders, such as multiple sclerosis (MS), are characterized by demyelination and the presence of inflammation in the central nervous system (CNS) leading to neuronal and/or myelin injury and loss and consequential neurological impairment. MS is the most common disabling neurological condition of European, North American, and other temperate climates. Currently approved front-line treatments for MS include interferon-β or glatiramer acetate. These agents are thought to act by shifting the immune balance toward an anti-inflammatory response.

### SUMMARY

Described herein are methods, compositions, and kits for treating, ameliorating, or for prophylactically addressing symptoms of a neuroinflammatory or demyelinating disorder in a mammal. For example, provided herein is a method of treating or ameliorating a neuroinflammatory or demyelinating disorder, such as multiple sclerosis, in a mammal that includes the steps of administering to the mammal a prolactin, a prolactin inducing agent, or a variant, fragment or analog of either prolactin or a prolactin inducing agent; and administering to the mammal an immunomodulator (e.g., interferon, COPAXONE®, CAMPATH®, MBP8929). Compositions and kits including prolactin and an immunomodulator are also provided. The composition is optionally a pharmaceutical composition for use in treating or ameliorating a neuroinflammatory or demyelinating disorder. Thus provided herein is a use for a composition including prolactin and an immunomodulator for the manufacture of a medicament for treating or ameliorating a neuroinflammatory or demyelinating disorder.

### DESCRIPTION OF DRAWINGS

FIG. 1 shows the treatment paradigm for the administration of prolactin, interferon-β, MOG and pertussis toxin.

FIG. 2 is a graph showing the mean clinical scores in EAE mice in the presence of vehicle, prolactin, interferon-β, or the combination of prolactin and interferon-β over three weeks. The combination of prolactin and interferon-β attenuated EAE clinical scores as compared to the effect with either agent alone and as compared to vehicle.

FIG. 3 is a histogram showing the sum of the clinical scores in EAE mice treated with vehicle, prolactin, interferon-β, or the combination of prolactin and interferon-β from FIG. 2. FIG. 3A shows the results at day 17 of the treatment period. FIG. 3B shows the results at day 21, 4 days after withdrawal of treatment. Values are means±standard errors of the means. Statistical analysis consisted of ANOVA with Tukey's multiple comparisons test.

### DETAILED DESCRIPTION

Prolactin has been demonstrated to increase neural stem cells, enhance neurogenesis, and increase oligodendrocyte precursor cells (OPCs) (see, e.g., U.S. Pat. No. 7,393,830, US Patent Application No. 2007/0098698). Prolactin also enhances remyelination after a toxin (lysolecithin)-mediated demyelination of the spinal cord (Gregg et al. 2007 White matter plasticity and enhanced remyelination in the maternal CNS, J. Neuroscience 27:1812-1823). However, there are suggestions in the literature that suggest prolactin may be pro-inflammatory in conditions such as experimental autoimmune encephalitis (EAE), an animal model of MS. Using the presently described methods and compositions, however, neither prolactin alone nor in combination with an immunomodulator, in an art-accepted animal model of a neuroinflammatory, demyelinating disease like multiple sclerosis exacerbates disease severity. In fact, prolactin and an immunomodulator (e.g., interferon-β) have a synergistic effect in reducing disease severity, as compared to either agent alone. Furthermore, the effect is maintained after withdrawal of the treatment. The combination of prolactin and interferon also modifies growth factor expression profile, as compared to either agent alone.

Provided herein are methods and compositions for treating or ameliorating disorders associated with neuroinflammation and/or demyelination, or for treating or ameliorating paralysis or dysfunction associated with such disorders. Also provided are methods for enhancing the formation of myelin, for enhancing the number of oligodendrocyte precursor cells in a mammal, for preventing demyelination, for enhancing the number of oligodendrocytes, for reducing neuronal or axonal loss, reducing the size or number of lesions, or for reducing inflammation. Further provided is a method of reducing the likelihood of or delaying relapse of a neuroinflammatory or demyelinating disease such as a relapsing form of MS. The methods and compositions relate to the use of a prolactin or a prolactin-inducing agent in combination with an immunomodulator. The combined administration of a prolactin or a prolactin-inducing agent and an immunomodulator has one or more combinatorial or synergistic effects. For example, the combination of prolactin and interferon has a synergistic effect in reducing the severity of clinical symptoms in an animal model of MS.

Immunomodulators include agents that affect the immune system by down-regulating immune destructive processes without eliminating the capacity to ward off infections. Examples of immunomodulators include interferon (interferon-β (interferon-β-1b (BETASERON® (Schering Aktiengesellschaft Corp., Berlin, Germany)); interferon β-1a (e.g., AVONEX® (Biogen Inc., Cambridge, Mass.), REBIF® (Ares-Serano, Aubonne Switzerland), CINNOVEX™ (CinnoGen Co., Tehran, Iran)); interferon-□ (INTRON®A (Schering Corp., Kenilworth, N.J.), ROFERON-A® (Hoffman-LaRoche, Inc., Nutley, N.J.))) and glatiramer acetate (COPAXONE (Teva Pharmaceuticals, Tikva, Israel)). Other possible immunomodulators include alemtuzumab (CAMPATH® (Genzyme, Cambridge, Mass.)); MBP8929 (BioMS Medical, Edmonton, Alberta, CA) mitoxantrone (e.g., NOVANTRONE® (Immunex, Seattle, Wash.)), natalizumab (e.g., TYSABRI® (Elan Pharma International Ltd., Claire, Ireland)), azathioprine (e.g., IMURAN® (Prometheus Lab, San Diego, Calif.)), methotrexate (e.g., RHEUMATREX® (Dava International, Inc. Fort Lee N.J.)), cyclosphosamide (e.g., CYTOXAN® (Mead Johnson & Co., Evansville, Ind.)), cyclosporine (e.g., SANDIMMUNE® (Novartis AG Corp, Basel Switzerland), cladribine (e.g., LEUSTATIN® (Johnson & Johnson Corp., New Brunswick, N.J.). By way of example, interferon-β is used throughout this disclosure as the immunomodulator. The immunomodulator can be an active fragment, variant, or analog of the immunomodulator (e.g., of interferon β). Note that for prolactin or interferon, human and non-human forms are included.

Optionally, the methods disclosed herein also include administering to the mammal an additional agent, including, for example, minocycline or an agent that promotes differentiation of oligodendrocytes. Examples of such agents include thyroid hormone in the T3 or T4 form and thyroid releasing hormone.

The agents or compositions used in the methods herein can be administered systemically (e.g., orally, parenterally (e.g., intravenously), intramuscularly, intraperitoneally, transdermally (e.g., by a patch), extracorporeally, topically, by inhalation, subcutaneously or the like), by administration into the central nervous system (e.g., into the brain (intracerebrally or intraventricularly) or spinal cord or into the cerebrospinal fluid), or any combination thereof. The interferon-β is administered systemically or into the central nervous system, and the prolactin or prolactin-inducing agent is administered systemically, or into the central nervous system. The mode of administration of the various agents can be the same, or different and used in any combination. Thus, for example, the interferon-β and the prolactin or prolactin-inducing agent can both be administered systemically or they can be administered by different methods (e.g., one by systemic administration and one by administration into the central nervous system). Where additional agents are administered, they can similarly be administered by the same method or by different methods than the interferon-β and/or the prolactin or prolactin inducing agent.

The dosage of the compositions or agents required will vary from subject to subject, depending on the species, age, weight, and general condition of the subject, the particular active agent used, its mode of administration and the like. For example, animal models for a variety of neuroinflammatory and/or demyelinating disorders (e.g., the EAE mouse model, cuprizone induced demyelination model and lysolecithin model) can be used to monitor levels of response. Clinical signs can be monitored and end point histopathological analyses of inflammation, demyelination and axonal disruption in the spinal cord can be determined as previously described in subjects with or in animal models of neuroinflammatory and/or demyelinating disorders. In addition, lymphocytes from the lymph node and the spinal cord at different times following MOG immunization can be measured in order to determine the ongoing inflammatory responses that are evoked by prolactin and/or an immunomodulator. Histological tests can be used to detect, for example, myelin, OPCs or oligodentrocyte numbers, remyelination, cytokine levels, lymphocyte number. Cell sorting can be performed to identify lymphocyte numbers in tissue samples. Imaging (e.g., MRI) and functional measurements (survival or clinical symptoms) can be used to monitor a subject's response to therapy. Furthermore, resected human brain samples (e.g., samples derived as a means to treat an otherwise intractable disorders) can be dissociated and cultured as neurospheres to study the effect of doses of prolactin and/or an immunomodulator on the subject's OPCs and their ability to enhance proliferation and self-renewal. Electrophysiology studies (e.g., evoked potentials and nerve conduction) can be utilized to monitor progress. For examples of neuropathological examinations, see, e.g., U.S. Patent Application No. 2007/023711; Schellenberg et al. (2007) Magnetic resonance imaging of blood-spinal cord barrier disruption in mice with experimental autoimmune encephalomyelitis, Magn. Reson. Med. 58:298-305; and Larsen et al. (2003) Matrix metalloproteinase-9 facilitates remyelination in part by processing the inhibitory NG2 proteoglycan, J. Neuroscience 23:11127-35. Brundula et al., Brain 125:1297, 2002; Giuliani et al., J Neuroimmunol 165:83, 2005

As a general rule, the dosage ranges for the administration of the compositions are those large enough to produce the desired effect in which the symptoms of the disease are affected. For example, functional improvement, histological improvement, gene expression, and the like can be affected and monitored to determine the desired outcome. The dosage should not be so large as to cause adverse side effects, such as unwanted cross-reactions and unwanted inflammatory reactions. Dosage can vary, but it should be noted that lower doses of the agents may be used in combination than either agent alone in view of the synergistic effects. Examples of human doses of prolactin include about 1-1000 μg/kg, about 10-100 μg/kg, and more particularly about 40-60 μg/kg (or any amount in between) by subcutaneous injection daily or any dose and mode of administration that achieves a prolactin blood level in the range of that measured in postpartum women, i.e. about 100-250 μg/l. In general, the doses of immunomodulator can be similar to or less than those used in monotherapy. Examples of human dosages of interferon-β include about 1-1000 μg or about 10-250 μg (or any amount in between) administered as follows, for example: intramuscularly once a week (e.g., about 1-100 μg or about 20-35 μg once weekly); subcutaneously three times per week (e.g., about 1-100 μg or about 10-50 μg three times a week); and subcutaneously on alternate days with about 20-500 μg (e.g., about 200-250 μg every other day). Examples of human doses of COPAXONE® include about 1-100 mg or about 10-100 mg subcutaneously daily (e.g., about 20 mg daily).

The agents can be administered in one or more dose administrations at least once daily, for one or more days (e.g., 1, 2, 3, 4, 5, 6, 7 days; 2, 3, 4 weeks; 2, 3, 4, 5, 6, 7, 8, 9, 10, 11, 12 months; 2 years, 3 years, 4 years, or longer). The agents are not necessarily administered on the same schedule. One agent can be administered daily and another agent less frequently, for example. Treatment optionally is semi-weekly, weekly, bi-weekly, semi-monthly, monthly, bimonthly, etc. Thus, the duration of treatment can optionally be for one or more days, weeks, months, or years, and can be sustained for the remaining lifetime of the subject.

The agents (e.g., the prolactin or prolactin-inducing agent, the interferon-β, and/or other agents) are optionally administered sequentially or concurrently. By concurrent administration is meant administration within the same composition or by administration in separate compositions in approximately the same period of time. By sequential administration is meant that the agents are administered in series with one or more intervening time periods between administration that can include minutes (1-60 or any amount in between), hours (1-24 or any amount in between), days (1-7 or any amount in between), or weeks (1-52 or any amount in between). Optionally, the prolactin or prolactin inducing agent is delivered first, or, alternatively, the interferon-β is administered first. By way of example, a first agent can be administered followed by a second agent with an interval between the administrations in which the first agent is physiologically effective or active during or overlapping with the period of physiological effectiveness or activity of the second agent.

Any one of the agents or combinations of agents herein can be administered along with an agent or under conditions that enhance passage of the agent or agents across the blood brain barrier. For example, a blood brain barrier permeabilizer can be utilized. Blood brain barrier permeabilizers are known in the art and include, by way of example, bradykinin and the bradykinin agonists described in U.S. Pat. Nos. 5,686,416; 5,506,206 and 5,268,164 (such as NH₂-arginine-proline-hydroxyproxyproline-glycine-thienylalanine-serine-proline-4-Me-tyrosineψ(-CH₂NH)-arginine-COOH). Alternatively, the molecules to be delivered can be conjugated to transport vectors including for example, the transferrin receptor antibodies (as described in U.S. Pat. Nos. 6,329,508; 6,015,555; 5,833,988 or 5,527,527), cationized albumin, insulin, insulin-like growth factors (IGF-I, IGF-II), angiotensin II, atrial and brain natriuretic peptide (ANP, BNP), interleukin I (IL-1), transferrin, cationized LDL, albumin or horseradish peroxidase coupled with polylysine, cationized albumin, cationized immunoglobulin, small basic oligopeptides (e.g., dynorphin analogue E-2078 or the ACTH analogue ebiratide), hexose moieties (e.g., glucose), monocarboxylic acids (e.g., lactic acid). The agents can also be delivered as a fusion protein comprising the molecule and a ligand that is reactive with a brain capillary endothelial cell receptor, such as the transferrin receptor (see, e.g., U.S. Pat. No. 5,977,307).

Optionally, the agents can be formulated, for example, in liposomes or can be PEGylated (i.e., covalent attachment of polyethylene glycol polymer chains to the agent or agents) according to methods available in the art. The liposomes may comprise one or more moieties which are selectively transported into specific cells or organs (targeting moieties), thus providing targeted drug delivery. Exemplary targeting moieties include folate, biotin, mannosides, antibodies, surfactant protein A receptor and gp120.

Also provided herein are methods of treating or ameliorating a neuroinflammatory or demyelinating disorder in a mammal and methods for enhancing the formation of myelin, for preventing demyelination, for enhancing the number of oligodendrocyte precursor cells, for enhancing the number of oligodendrocytes, for reducing neuronal or axonal loss, reducing the size or number of lesions, or for reducing inflammation in a mammal, which include the steps of administering to the mammal a prolactin or a polypeptide having at least 80-99% sequence identity (or any sequence identity between 80 and 99%) with a prolactin or a prolactin-inducing agent and administering to the mammal an interferon-β.

Those of skill in the art readily understand how to determine the sequence identity of two polypeptides. For example, the identity can be calculated after aligning the two sequences so that the identity is at its highest level. Published algorithms can be used to determine the percentage of identity. Optimal alignment of sequences for comparison may be conducted by published algorithms (e.g., Smith and Waterman, Adv. Appl. Math. 2: 482 (1981), Needleman and Wunsch, J. Mol. Biol. 48: 443 (1970), Pearson and Lipman, Proc. Natl. Acad. Sci. U.S.A. 85: 2444 (1988)) or by computerized implementations of algorithms (e.g., GAP, BESTFIT, FASTA, and TFASTA in the Wisconsin Genetics Software Package, Genetics Computer Group, 575 Science Dr., Madison, Wis.).

As described above for methods using a prolactin or a prolactin inducing agents, the immunomodulatory agent is optionally interferon-β, and the methods using a polypeptide having at least 80-99% sequence identity with a prolactin or a prolactin-inducing agent can further include administering to the subject other agents (e.g., triiodothyronine or other agent that promotes differentiation of oligodendrocytes or minocycline). The modes of administration and the timing of the administration are also as described above.

The steps of the methods taught herein are also useful in preventing or delaying the onset of symptoms associated with a neuroinflammatory or demyelinating disorder. Thus, a subject at risk for a neuroinflammatory or demyelinating disorder or having an asymptomatic neuroinflammatory or demyelinating disorder is administered a prolactin or prolactin inducing agent and administered an interferon-β (and optionally other agents) according to the teachings herein. For example, a subject with a brain injury would be known to be at risk for neuroinflammation and/or demyelination and would benefit from these treatment regimes. Risk for neuroinflammation or demyelination or need for prophylactic treatment can be based on MRI or by presymptomatic indications appreciated by one of skill in the art.

A composition comprising a prolactin or a prolactin-inducing agent and an immunomodulator, such as interferon (e.g., interferon-β), is also provided. This composition is for use in treating or ameliorating a disorder associated with neuroinflammation or demyelination. The composition can further comprise a pharmaceutically acceptable carrier and/or other agents (e.g., agents that enhance blood brain barrier permeability). By pharmaceutically acceptable is meant a material that is not biologically or otherwise undesirable. Thus, the material may be administered to a subject, without causing unacceptable biological effects or unacceptable interactions with any of the other components of the pharmaceutical composition in which it is contained. The carrier would naturally be selected to minimize any degradation of the prolactin, prolactin inducing agent, or interferon-β and to minimize any adverse side effects in the subject. Suitable carriers and their formulations are described in Remington: The Science and Practice of Pharmacy (21st ed.) ed. University of the Sciences in Philadelphia, Lipincott Williams & Wilkins 2005. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8.5, and more preferably from about 7.8 to about 8.2. Further carriers include sustained release preparations such as semipermeable matrices of solid hydrophobic polymers containing the antibody, which matrices are in the form of shaped articles, e.g., films, liposomes or microparticles. Certain carriers may be more preferable depending upon, for instance, the route of administration and concentration of composition being administered. Pharmaceutical compositions may include carriers, thickeners, diluents, buffers, preservatives, surface active agents and the like in addition to the molecule of choice. Pharmaceutical compositions may also include one or more active ingredients such as antimicrobial agents and the like.

Also provided herein is a kit for treating or ameliorating a disorder associated with neuroinflammation or demyelination in a subject. The kit includes a prolactin or a prolactin inducing agent and an immunomodulator (e.g., interferon-β) in one or more containers. The kit optionally comprises at least one container with both the prolactin or prolactin-inducing agent and the interferon-β. Thus, the kit may include a container with the prolactin without the interferon-β, may include a container with an interferon-β without the prolactin, may include a container with both the prolactin and the interferon-β, or may contain combinations thereof. Any of the kits provided herein optionally include instructions for administering the agents or compositions to a subject and/or include at least one device for administering the agents or compositions to the subject.

Also provided herein is the use of a composition comprising a prolactin or a prolactin inducing agent and an immunomodulator (e.g., interferon-β) for the manufacture of a medicament for treating or ameliorating a disorder associated with neuroinflammation and/or demyelination.

It should be noted that prolactin is recited throughout by way of example. Also useful in the methods and compositions are variants, fragments, analogs of a prolactin or a prolactin-inducing agent, wherein the variant, fragment or functional analog has biological effects comparable to or better than the biological effects of prolactin.

The variants, fragments, and analogs can be obtained, for example, by extraction from a natural source (e.g., a mammalian cell), by expression of a recombinant nucleic acid encoding the polypeptide (e.g., in a cell or in a cell-free translation system), or by chemically synthesizing the polypeptide. In addition, polypeptide fragments may be obtained by any of these methods, or by cleaving full length polypeptides.

Polypeptide variants that share substantial sequence identity with a naturally occurring prolactin (e.g., 70-99% sequence identity or any amount of sequence identity between 70 and 99%) can be used in the methods and compositions taught herein so long as the polypeptide variants have one or more biological activities of a prolactin (e.g., binding to a prolactin receptor). Such prolactin variants include deletional, insertional, or substitutional mutants of the native prolactin. Insertions include amino and/or carboxyl terminal fusions as well as intrasequence insertions of single or multiple amino acid residues. Deletions are characterized by the removal of one or more amino acid residues from the protein sequence. Substitutions can comprise one or more conservative amino acid substitutions, one or more non-conservative amino acid substitutions, or any combination thereof. These variants ordinarily are prepared by site specific mutagenesis of nucleotides in the DNA encoding the polypeptide, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Substitutions, deletions, and insertions may be combined to arrive at a final construct.

Fragments of a prolactin can also be used instead of a prolactin in the methods and compositions taught herein. For example, a fragment of a prolactin that includes all or a binding portion of the prolactin receptor binding region is useful in the methods and compositions.

Functional analogs (either naturally occurring or non-naturally occurring) of a prolactin can also be used. For example, the functional agonist may be an activating amino acid sequence disclosed in U.S. Pat. No. 6,333,031 for the prolactin receptor; a metal complexed receptor ligand with agonist activities, for the prolactin receptor (U.S. Pat. No. 6,413,952); G120RhGH which is an analog of human growth hormone but acts as a prolactin agonist (Mode, et al Endocrinology 137:447 (1996)); or a ligand for the prolactin receptor as described in U.S. Pat. Nos. 5,506,107 and 5,837,460. Specifically, naturally occurring prolactin variants, prolactin-related protein, S179D-human prolactin (Bernichtein, S. et al. Endocrinology 142:3950 (2001)), prolactins from various mammalian species, including but not limited to, human, other primates, rat, mouse, sheep, pig, cattle, and the prolactin mutants described in U.S. Pat. Nos. 6,429,186 and 5,995,346 can be used in the methods and compositions herein.

Instead of or in addition to a prolactin, a prolactin inducing agent can be administered to increase the level of prolactin in the subject. By way of example, prolactin releasing peptide and naturally-occurring or non-naturally-occurring variants, as well as functional fragments or analogs of prolactin releasing peptide can be used in the methods and compositions herein.

By subject is meant any individual including a mammal. For example, subjects include primates, rodents, felines, canines, domestic livestock (such as cattle, sheep, goats, horses, and pigs), and humans.

A neuroinflammatory disorder is a disease or condition caused by or associated with inflammation of the nervous system. Neuroinflammatory disorders can be associated with demyelination. A demyelinating disorder is caused by or associated with demyelination or dysmyelination in the central or peripheral nervous system. Examples of neuroinflammatory or demyelinating disorders include multiple sclerosis (including the relapsing and chronic progressive forms of multiple sclerosis and acute multiple sclerosis), neuromyelitis optica (Devic's disease), optic neuritis, diffuse cerebral sclerosis (including Shilder's encephalitis periaxialis diffusa and Balo's concentric sclerosis), transverse myelitis, and acute disseminated encephalomyelitis (e.g., occurring after measles, chickenpox, rubella, influenza or mumps; or after rabies or smallpox vaccination), necrotizing hemorrhagic encephalitis (including hemorrhagic leukoencephalitis), leukodystrophies (including Krabbe's globoid leukodystrophy, metachromatic leukodystrophy, adrenoleukodystrophy, Canavan's disease and Alexander's disease), CNS injury (e.g., spinal cord injury, head injury, or stroke), age-related dementia, depression, and bipolar disorders. Other examples include Guillain-Bane syndrome and chronic inflammatory demyelinating polyneuropathy (CIDP).

Treating or ameliorating means the reduction or complete removal of one or more symptoms or signs of a disease or medical condition or a delay in the onset or reoccurrence of symptoms or signs (including neuropathological signs) of the disease or medical condition. An effective amount is an amount of a therapeutic agent sufficient to achieve the intended purpose. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art and as taught in the Examples.

The effectiveness of a dosing regime can be performed using methods taught herein or methods known to one of skill in the art. For example, remyelination can be assessed using histochemical techniques, neurophysiological studies (nerve conduction studies), MRI analysis, clinical assessments, and the like. See, e.g., Gregg et al. (2007) White matter plasticity and enhanced remyelination in the maternal CNS, J. Neuroscience 27:1812-1823; Giuliani et al. (2005) Effective combination of minocycline and interferon-β in a model of multiple sclerosis, J. Neuroimmunology 165:83-91; US Patent App. No. 2007/0238711; Giuliani et al. (2005) Additive effect of the combination of glatiramer acetate and minocycline in a model of MS, J. Neuroimmunology 158:213-221, all of which are incorporated herein by reference in their entireties for the methods and composition taught therein.

When relative terms like enhance, increase, decrease, reduce, and the like are used herein they are generally used in reference to a control level. For example, a control level can be that of a different subject lacking a disease or condition or lacking an experimental variable, such as treatment with an agent, or a control level can be from the same subject before onset or after cure of the disease or condition or before or after exposure to an experimental variable.

The compositions and methods disclosed herein can be used in various combinations. Thus, it is understood that when combinations, subsets, interactions, steps, groups, etc. of these materials or methods are disclosed that, while specific references to each various individual and collective combination may not be explicitly disclosed, each is contemplated and described herein. For example, if a combination of a prolactin and a blood brain barrier permeabilizer is recited and a combination of a prolactin variant and an interferon-β is recited, disclosed and discussed are every combination of each of these agents, unless specifically indicated to the contrary.

The present methods, kits and compositions are more particularly described in the following examples, which are intended as illustrative only since numerous modifications and variations therein will be apparent to those skilled in the art.

### EXAMPLES

### Example 1

### Behavioral Effects of Prolactin and an Immunomodulator in Mice with Experimental Autoimmune Encephalomyelitis (EAE)

C57BL/6 female mice were purchased from Charles River and were of 6-8 weeks upon arrival at the University of Calgary. After a week of acclimatization at the Animal Resource Facility at the University of Calgary, mice were immunized with 50 μg of myelin oligodendrocyte glycoprotein (MOG, peptide 35-55) emulsified in Complete Freund's adjuvant (CFA) (Fisher, Mich. USA) supplemented with 4 mg/ml of Mycobacterium tuberculosis; this preparation was injected as a 100 μl suspension in the flank. The day of MOG immunization was referred to as day zero of experiment. In addition, mice received 2 injections of pertussis toxin (PTX) (List Biological labs, Hornby, ON) by intraperitoneal route (300 ng per injection) on Days 0 and 2. Mice were allowed free access to food and water at all time points and were handled in accordance with the policies outlined by the Canadian Council for Animal Care. Two experiments were conducted.

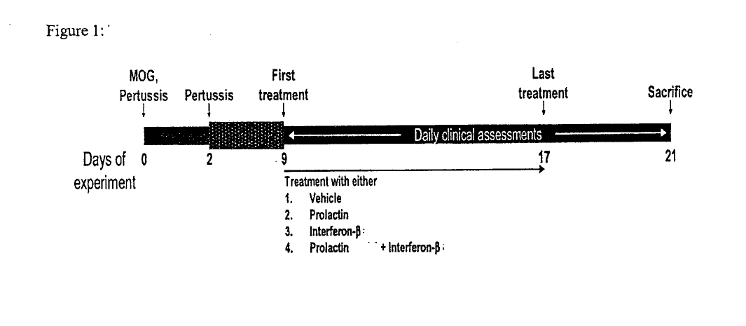

This experiment lasted over 21 days where at day 9 following MOG immunization, treatment was initiated (see FIG. 1). This consisted of either recombinant murine prolactin (Harbor-UCLA, Torrance, Calif.) or recombinant murine interferon-β (PBL Biomedical Laboratories, Piscataway, N.J.) given alone or in combination, or vehicle control. In essence, there were 4 groups of mice:

1) The prolactin alone group (n=6 mice) received 100 μl of prolactin by the intraperitoneal route, equivalent to 20 μg/mouse. Treatment was once per day from days 9 to 17;

2) The interferon-β alone group (n=4) received 200 μl of interferon-β by the subcutaneous route, equivalent to 20,000 IU/mouse, every other day, from days 9 to 17;

3) The prolactin and interferon-β combination group (n=4) received the above doses at the above frequency (once a day for prolactin and every other day for interferon-β using the above routes from days 9 to 17;

4) The vehicle group (n=7) from days 9 to 17 received 100 μl of the vehicle (saline) used to dissolve prolactin every day by intraperitoneal injection, and 200 μl of the vehicle (phosphate-buffered saline containing 0.1% bovine serum albumin) used to dilute interferon-β from by the subcutaneous route.

Mice were evaluated for clinical signs daily from days 9 to 21. Animals were assessed using a 15-point disease score scale (Giuliani et al., Additive effect of the combination of glatiramer acetate and minocycline in a model of MS, J Neuroimmunol 158:213-221, 2005; Weaver et al., An elevated matrix metalloproteinase in experimental autoimmune encephalomyelitis is protective by affecting Th1/Th2 polarization, FASEB J 19:1668-1670, 2005) that differentiates individual limb and tail disability. The 15-point scale (Table 1) ranges from 0 to 15 and is the sum of the disease state for the tail (which is scored from 0-2) and all 4 limbs (each limb is scored from 0-3). Based on this scoring system a fully quadriplegic mouse attains a score of 14 and mortality is given a score of 15.

TABLE 1
The 15-point scoring scale

| Score | Symptoms |
|---|---|
| Tail | |
| 0.5 | Only half tail limp |
| 1.0 | Complete limp tail, animal can lift it up once in a while |
| 2.0 | Complete limp tail, no tension, animal cannot lift it up at |
| | all |
| Limbs (Hind | |
| L and R) | |
| 1.0 | Weakness of limb, no tension detected when pushed upon, |
| | will not use to hold onto objects, toes may be |
| | slightly curved |
| 2.0 | Duck-like splayed feet walk, lowered hind quarter |
| 3.0 | Foot completely twisted, and may drag behind animal |
| | when walking forward |
| Limbs (Fore | |
| L and R) | |
| 1.0 | Weakness of limb, no tension detected when pushed upon, |
| | will not use to hold onto objects, fingers may be |
| | slightly curved |
| 2.0 | No grasp at all |
| 3.0 | No tension detected when pushed upon, will not use to |
| | hold onto objects, fingers may be slightly curved, |
| | at this stage the animal will be very paralyzed, its |
| | back will be curved inward, and it will have |
| | difficulty breathing, eating, or crawling around |

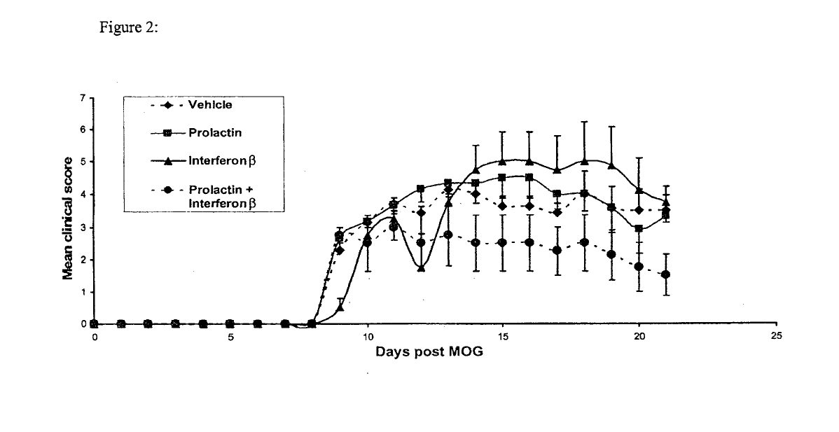

The results are shown in FIG. 2. Mice in all groups generally succumbed to signs of EAE at day 9 following MOG. In contrast to the other treatment groups, the mice given prolactin and interferon-β in combination did not increase in level in disability from that attained at day 9. Overall, mice on the combination treatment had a reduced level of clinical score from the other 3 groups and this difference appeared apparent from days 13 to the termination of the experiment at day 21 (FIG. 2).

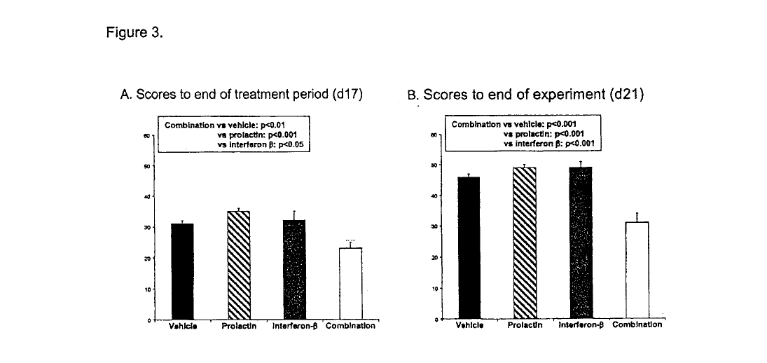

To compare statistically across the groups, the sum of score was plotted for each mouse. The sum of score is the cumulative total score of each mouse, when the daily score for that mouse is added over the course of the experiment. Thus, the sum of score represents the overall burden of clinical disease per mouse. The sum of scores was calculated for mice up to day 17 (at end of treatment period) or to day 21 (when mice were sacrificed, and where there were no treatment between days 18 to 21). FIG. 3 shows that there was a statistical difference between the combination group compared to controls. Collectively, the results indicate that the combination of prolactin and interferon-β resulted in lowering disease severity. Furthermore, prolactin, at least in combination with an immunomodulator does not exacerbate the disorder, and the combination therapy actually had a sustained benefit after the withdrawal of treatment.

### Example 2

### RNA Analysis Related to the Effect of Prolactin and an Immunomodulator in Mice with Experimental Autoimmune Encephalomyelitis (EAE)

The same treatment protocol described in Example 1 was repeated, except that mice were sacrificed on day 17 and the clinical symptoms of the EAE using a different batch of pertussis toxin were worse than in the experiment described above. Although the animals in this treatment groups did not show statistically significant improvement in clinical symptoms as demonstrated above, they did show a difference in gene expression.

For purposes of the gene experiments, the spinal cord was removed, the lumbar-sacral cord was dissected and total RNA was isolated using the RNeasy kit protocol (QIAGEN). One μg of RNA was reversed transcribed to cDNA and real time polymerase chain reaction (PCR) for multiple growth factors was simultaneously performed according to manufacturer's protocol. The Mouse Growth Factor array # PAMM-041A (RT2 profiler PCR array, SuperArray Bioscience Corporation, Fredrick, Md.) was used.

This consisted of 84 genes encoding growth factors plus 5 housekeeping genes and 7 control wells (to control for mouse genomic DNA contamination, reverse transcription control, an positive PCR control). Cycling parameters were 95° C. for 10 minutes, followed by 40 cycles of 95° C. for 15 seconds and 60° C. for 1 minute. mRNA expression of each gene was normalized using the expression of 5 housekeeping genes included in the gene array. Analysis was performed using the web based software provided by the company, and fold changes were obtained using the vehicle group as control. There were 3 mice per experimental group and each mouse was assessed individually in the gene array. Moreover, statistical analysis of the 3 mice per group, compared to the animals in the vehicle allowed particular growth factors to be detected as being elevated in the experimental groups. Note only genes that were upregulated at least 1.5 fold or were downregulated at least 0.6 fold, and in a statistically significant manner across the experimental group compared to the EAE vehicle mice were considered. Of 84 growth factor genes in the PCR array, only 1 growth factor (Interleukin-11) was found elevated in the prolactin alone group, and 1 was reduced (Interleukin-4) compared to vehicle-treated group afflicted with EAE. In the interferon-β-only group, two growth factors (—Fibroblast growth factor-10 and Insulin-like growth factor-2) are elevated, while three genes were reduced (Interleukin-6, Leukemia inhibitory factor, and Transforming growth factor-β1). Interestingly, the combination of prolactin and interferon-β resulted in a different profile of growth factor expression compared to either prolactin or interferon-β alone. See Table 2

TABLE 2
Upregulated (+) and Downregulated (−) Growth Factor Genes with
Prolactin, Interferon-β or Prolactin + Interferon-β

| | | Treatment | Prolactin + |
|---|---|---|---|
| Gene | Prolactin | Interferon-β | Interferon-β |
| Interleukin-4 | −0.6 (p < 0.05) | | |
| Interleukin-6 | | −0.5 (p < 0.01) | |
| Leukemia inhibitory | | −0.5 (p < 0.05) | |
| factor | | | |
| Transforming growth | | −0.6 (p < 0.05) | |
| factor-β1 | | | |
| Interleukin-11 | +1.7 (p < 0.01) | | |
| Fibroblast growth | | +1.6 (p < 0.02) | |
| factor-10 | | | |
| Insulin-like growth | | +1.7 (p < 0.04) | |
| factor 2 | | | |
| Anti-Mullerian | | | +1.5 (p < 0.03) |
| hormone | | | |
| Artemin | | | +2.1 (p < 0.05) |
| Bone morphogenetic | | | +1.5 (p < 0.03) |
| protein 10 | | | |
| Bone morphogenetic | | | +1.5 (p < 0.03) |
| protein 8b | | | |
| Colony stimulating | | | +3.0 (p < 0.03) |
| factor 2 | | | |
| Epiregulin | | | +1.5 (p < 0.03) |
| Fibroblast growth | | | +1.5 (p < 0.03) |
| factor 15 | | | |
| Fibroblast growth | | | +1.5 (p < 0.03) |
| factor 6 | | | |
| Fibroblast growth | | | +1.5 (p < 0.03) |
| factor 8 | | | |
| Leptin | | | +1.5 (p < 0.03) |
| Neurotrophin ⅘ | | | +1.5 (p < 0.03) |
| S100 calcium binding | | | +1.7 (p < 0.05) |
| protein A6 | | | |

Overall, the results indicate that the combination of prolactin and interferon-β provides a combined effect of modifying the growth factor expression profile in an accepted animal model of a neuroinflammatory, demyelinating disease.

### Example 3

### Histological Studies Related to the Effect of Prolactin and an Immunomodulator in Mice with Experimental Autoimmune Encephalomyelitis (EAE)

EAE mice are developed as described in the previous Examples. Lymphocytes from the lymph node and the spinal cord are obtained at different times following MOG immunization to determine the ongoing inflammatory responses using standard histological techniques. To determine whether remyelination occurs in EAE mice treated with a combination of prolactin and an immunomodulator, animals are sacrificed at various time points before and during the disease progression, including, for example, 5 days after peak disease has been attained in the control group. The spinal cord is processed to determine the number of OPCs and remyelination is assessed using techniques described above and known in the art, focusing on the dorsal column of the lumbar sacral cord, where injury in EAE is found consistently.

Optionally OPCs are labeled with antibodies to PDGFRα, oligodendrocytes are labeled using antibodies to GSTpi as know in the art. By staining and counting multiple sections per lumbar or sacral segment, and by counting multiple lumbar sacral segments, the number of OPCs and oligodendrocyte numbers are counted in particular treatment on control groups. Moreover, by staining for myelin using luxol fast blue, eriochrome cyanine or myelin basic protein stains, and by obtaining the volume of demyelination that remains in the lumbar sacral cord, the extent of remyelination is inferred (animals that remyelinate better have less remaining volume of demyelination). Finally, MRI of the spinal cord of the EAE mice is performed, and the volume of T2 lesions is monitored in groups of mice at a particular time point (e.g. 5 days after peak clinical disease), or in each mouse longitudinally over time (e.g., before clinical signs, at peak clinical disease, and at 5 and 30 days after peak). A resolving T2 lesion volume over time indicates recovery in the form of remyelination, and the spinal cord of these mice is then optionally taken for ultrastructural study of g-ratios (ratio of the diameter of axon±myelin, over diameter of the axon) for confirmation of remyelination.

Histology is also analyzed by hematoxylin-eosin for evidence of inflammation, Luxol fast blue for evidence of demyelination and Bielchowsy silver stain to identify axons as previously described (Giuliani et al., J. Neuroimmunol 158:213-221; Giuliani et al., J Neuroimmunol 165:83, 2005.

Throughout this application, various publications are referenced. The disclosures of these publications in their entireties are hereby incorporated by reference into this application in order to more fully describe the state of the art to which this invention pertains. The following references, for example, are incorporated by reference in their entireties: Gregg, C. et al. (2007) White matter plasticity and enhanced remyelination in the maternal CNS. J. Neurosci. 27 (8): 1812; Holstad, M., Sandler, S. (1999) Prolactin protects against diabetes induced by multiple low doses or streptozotocin in mice J. Endocrinol. 163 (2): 229; Kelley, K W et al (2007) Protein Hormones and Immunity. Brain Behav. Immun. 21(4): 384; Riskind, P N et al. (1991) The role of prolactin in autoimmune demyelination: suppression of experimental allergic encephalomyelitis by bromocriptine. Ann Neurol. 29 (5): 542; Tsutsui, S. et al. (2005) RON-regulated innate immunity is protective in an animal model of multiple sclerosis. Ann. Neurol. 57 (6): 883; Weaver, A. et al. (2005) An elevated matrix metalloproteinase (MMP) in an animal model of multiple sclerosis is protective by affecting Th1/Th2 polarization. FASEB J. 19 (12). Furthermore US Patent Publication No. 2007-0098698-A1 is incorporated herein by reference in its entirety, especially for methods and compositions related to prolactin and prolactin inducing agents, methods of screening for effects related to the same, and the like.

A number of embodiments of the methods and compositions have been described. Nevertheless, it will be understood that various modifications may be made without departing from the spirit and scope of the invention. For example, the use of prolactin inducing agents instead of prolactin can be used in embodiments in which only prolactin is recited. Accordingly, other embodiments are within the scope of the following claims.

1. A method of treating or ameliorating a neuroinflammatory or demyelinating disorder in a mammal, comprising:
a. administering to the mammal a prolactin or a prolactin inducing agent; and
b. administering to the mammal an interferon.

2. The method of claim 1, wherein the neuroinflammatory or demyelinating disorder is selected from the group consisting of multiple sclerosis, neuromyelitis optica, optic neuritis, diffuse cerebral sclerosis, transverse myelitis, acute disseminated encephalomyelitis, and central nervous system injury.

3. The method of claim 2, wherein the disorder is multiple sclerosis.

4. The method of claim 1, wherein the interferon is interferon-β.

5. A method of treating or ameliorating a neuroinflammatory or demyelinating disorder in a mammal, comprising:
a. administering to the mammal a variant of a prolactin or a variant of a prolactin inducing agent having at least 90% sequence identity with the prolactin or the prolactin inducing agent; and
b. administering to the mammal an interferon.

6. The method of claim 5, wherein the neuroinflammatory or demyelinating disorder is selected from the group consisting of multiple sclerosis, neuromyelitis optica, optic neuritis, diffuse cerebral sclerosis, transverse myelitis, acute disseminated encephalomyelitis, and central nervous system injury.

7. The method of claim 6, wherein said disorder is multiple sclerosis.

8. The method of claim 5, wherein the interferon is interferon-β.

9. A composition comprising prolactin and an interferon.

10. The composition of claim 9, wherein the interferon is interferon-β.

11. A kit for treating or ameliorating a neuroinflammatory or demyelinating disorder in a subject, comprising prolactin and an interferon in one or more containers.

12. The kit of claim 11, wherein the interferon is interferon-β.

13. The kit of claim 11, wherein the kit comprises at least one container with a combination of prolactin and the interferon.

14. The kit as in any one of claims 11, wherein the kit further comprises instructions for administering prolactin and the interferon to a subject.

15. The kit as in any one of claims 11, wherein the kit further comprises at least one device for administering prolactin or the interferon to a subject.

16. A pharmaceutical composition comprising prolactin and an interferon for use in treating or ameliorating a neuroinflammatory or demyelinating disorder.

17. The pharmaceutical composition of claim 16, wherein the neuroinflammatory or demyelinating disorder is selected from the group consisting of multiple sclerosis, neuromyelitis optica, optic neuritis, diffuse cerebral sclerosis, transverse myelitis, and acute disseminated encephalomyelitis, and central nervous system injury.

18. The pharmaceutical composition of claim 16, wherein the neuroinflammatory or demyelinating disorder is multiple sclerosis.

19. The pharmaceutical composition of claim 16, wherein the interferon is interferon-β.

20. Use of a composition comprising prolactin and an interferon for the manufacture of a medicament for treating or ameliorating a neuroinflammatory or demyelinating disorder.

21. The use of claim 20, wherein the neuroinflammatory or demyelinating disorder is selected from the group consisting of multiple sclerosis, neuromyelitis optica, optic neuritis, diffuse cerebral sclerosis, transverse myelitis, acute disseminated encephalomyelitis, and central nervous system injury.

22. The use of claim 20, wherein the neuroinflammatory or demyelinating disorder is multiple sclerosis.

23. The pharmaceutical composition of claim 20, wherein the interferon is interferon-β.

24. The method of claim 1, wherein prolactin is administered to the mammal.

25. The method of claim 5, wherein prolactin is administered to the mammal.
